(19) United States
(12) Patent Application Publication
(10) Pub. No.: US 2003/0023291 A1
(43) Pub. Date: Jan. 30, 2003

(54) Medical treatment device for a breast

(76) Inventor: Milene H. Hanner, Arnold, MD (US)

Correspondence Address: William L. Klima, A PROFESSIONAL LAW CORPORATION, Patent, Trademark & Copyright Law, 2046-C JEFFERSON DAVIS HIGWAY, STAFFORD, VA, 22554, US

(21) Appl. No.: 10/195,121

(22) Filed: Jul. 15, 2002

Related U.S. Application Data
(63) Continuation-in-part of application No. 09/533,589, filed on Mar. 23, 2000, now Pat. No. 6,419,691.

(51) Int. Cl. A61F 7/00

(52) U.S. Cl. 607/108; 607/112

(57) ABSTRACT

The present invention is directed to a medical device for treating a breast. Specifically, the medical device according to the present invention can apply heat or cold for treating the breast, and is configured to conform closely with the shape of the breast.

### RELATED APPLICATION

This application is a continuation-in-part (CIP) of the following U.S. Pat. Application:

1) “Thermal Energy Therapy”, Ser. No.: 09/533,589, filed on Mar. 23, 2000 (pending);

This application is all fully incorporated herein by reference.

### FIELD OF THE INVENTION

The present invention is related to the therapeutic application of thermal energy, specifically, heat or cold, to a human or animal. More specifically, the present invention is directed to a medical device for treatment of a human or animal breast.

### BACKGROUND OF THE INVENTION

There are many devices available for conducting thermal therapy. Some of these devices are extremely simple, for example, many households maintain an ice pack characterized by a gel filled bag in the freezer. Some of these devices are extremely complex, for example, some devices circulate a temperature controlled medium throughout a jacket or housing which enshrouds an anatomical treatment site.

Providing a device which effectively applies thermal energy to the body of a human has become particularly challenging due to the ever increasing demand to control costs in the health services industry. Health service institutions are now, more than ever before, being forced to purchase devices which are cheaper and unfortunately, less effective. There is thus an urgent need for devices and methods of manufacturing devices for applying thermal energy to a human or animal which provide increased effectiveness without increased cost.

Currently, there exists no medical device for effective treatment of the breast, especially after breast surgery or during breast feeding. Specifically, there exists no devices that closely conform with the breast and can apply heat or cold in an effective manner.

The present invention is configured to provide effective treatment of the breast, and overcome past difficulties with effective treatment thereof.

### SUMMARY OF THE INVENTION

A first object of the present invention is to provide a device and method of manufacturing a device for applying thermal energy to a human or animal.

A second object of the present invention is to provide an improved device and an improved method of manufacturing a device for applying thermal energy to a human or animal.

A third object of the present invention is to provide a device which applies thermal energy to an anatomical site precisely, for example, to prevent unnecessary application of such energy to portions of the body which do not require such treatment.

A fourth object of the present invention is to provide a device for applying thermal energy which is comfortable for the wearer.

A fifth object of the present invention is to provide a device for applying thermal energy which is easily worn under clothing.

A sixth object of the present invention is to provide a device for applying thermal energy which maintains a uniform dispersion of medium throughout the device.

A seventh object of the present invention is to provide a device for applying thermal energy which is easily secured to the anatomical site where thermal energy would be therapeutic, for example, a device which is provided with straps which may be connected or adjusted with one hand.

An eighth object of the present invention is to provide a device for applying thermal energy which is configured to apply thermal energy to a specific anatomical site, for example, the shoulder, knee, ankle, or breast.

A ninth object of the present invention is to provide a device for applying thermal energy which is beneficial to manufacture, for example, simple in design, easy to assemble, and cost effective.

A tenth object of the present invention is to provide a device for applying thermal energy to a human or animal including a deformable and resilient first layer sealingly connected to a deformable and resilient second layer along a common perimeter, the first layer further sealingly connected to the second layer along a plurality of curvilinear paths within the perimeter, the paths defining a plurality of channels between the first layer and the second layer, the paths being configured to allow articulation between adjacent channels, each channel having at least one end defined by the common perimeter, the at least one end of each channel being directly adjacent at least one end of another channel and, each channel being at least partially filled with a medium.

An eleventh object of the present invention is to provide a method of manufacturing a device for applying thermal energy to a human or animal including the steps of sealingly connecting a deformable and resilient first layer to a deformable and resilient second layer along a common perimeter, maintaining a pre-selected section of the common perimeter open such that the first and second layers remain independent along the pre-selected section of the common perimeter, sealingly connecting the first layer to the second layer along a plurality of curvilinear paths within the perimeter to form a plurality of channels within the perimeter having at least one open end along the pre-selected section of the common perimeter, holding the first and second layers such that the open pre-selected section of the common perimeter is oriented substantially vertically, filling each channel with a medium, and, sealingly connecting the first layer to the second layer along the open pre-selected section of the common perimeter.

The present invention provides a device for applying therapeutic thermal energy to the body of a human or animal. In a preferred embodiment, the present invention provides a device which includes a first layer and a second layer which are sealingly connected along a common perimeter and along a plurality of curvilinear paths within the perimeter, the paths defining a plurality of channels between the first layer and the second layer, the paths being configured to allow articulation between adjacent channels, each channel having at least one end defined by the common perimeter, the at least one end of each channel being directly adjacent at least one end of another channel and, each channel being at least partially filled with a medium.

The present invention further provides a method of manufacturing a device for applying thermal energy to a human or animal including the steps of sealingly connecting a deformable and resilient first layer to a deformable and resilient second layer along a common perimeter, maintaining a pre-selected section of the common perimeter open such that the first and second layers remain independent along the pre-selected section of the common perimeter, sealingly connecting the first layer to the second layer along a plurality of curvilinear paths within the perimeter to form a plurality of channels within the perimeter having at least one open end along the pre-selected section of the common perimeter, holding the first and second layers such that the open pre-selected section of the common perimeter is oriented substantially vertically, filling each channel with a medium, and, sealingly connecting the first layer to the second layer along the open preselected section of the common perimeter.

The present invention essentially provides a device which is extremely inexpensive to manufacture and at the same time provides many benefits over conventional means for applying thermal energy to the body. For example, devices according to the present invention provide a uniform application of medium over the treatment site, are easily secured to the body with optional straps, and may easily be worn under clothing. Moreover, devices according to the present invention easily adapt to the surface contours of the treatment site and some preferred embodiments of the device are directed specifically to the application of thermal energy, either hot or cold, to the shoulder, knee, ankle and breast. In sum, the present invention provides an improvement to the art of therapeutic thermal energy application.

### Medical Device For Treating A Breast

An object of the present invention is to provide a medical device for treating a human or animal breast.

A second object of the present invention is to provide a medical device configured to accommodate or conform with the shape of a breast.

A third object of the present invention is to provide a medical device for applying heat or cold to a breast.

A fourth object of the present invention is to provide a medical device for treating a breast, including a pair of sheets of deformable material connected together by at least one curvilinear seam.

A fifth object of the present invention is to provide a medical device for treating a breast including a pair of deformable sheets connected together by at least one curvilinear seam defining at least one curvilinear passageway.

A sixth object of the present invention is to provide a medical device for treating a breast including a pair of deformable sheets connected together by at least one curvilinear seam defining at least one curvilinear passageway, further including at least one additional seam provided in at least one passageway.

The present invention is directed to a medical device for treating a human or animal breast.

The medical device according to the present invention is preferably configured to conform with the shape of a breast. In one embodiment, the medical device is configured to treat a single breast, and in another embodiment the medical device is configured to treat a pair of breasts simultaneously.

The medical device according to the present invention is configured to deform and closely wrap around a breast. In a preferred embodiment, the medical device is made from a pair of layers connected together to form at least one curvilinear seam. The connection can be made by welding (e.g. heat welding, friction welding), adhesive bonding, fusion, mechanical fastener, sewing or any other suitable means or methods of connecting layers together while making the device leak proof. The curvilinear shape of the at least one seam and at least one passageway enhances the deformable nature of the medical device so as to closely conform with the shape of a breast to provide effective treatment thereof. It is important that the medical device makes as close a contact as possible with the contours of the breast to provide effective thermal heat transfer by conductive heat transfer.

Preferably, the medical device according to the present invention for treating a breast has a substantially circular outer perimeter defined by a perimeter seam. Further, the medical device is preferably provided with at least one notch in or through one or both layers of the sheet material to enhance the deformable nature of the device itself. In a more preferred embodiment, the notch extends from the outer perimeter into a center portion of the medical device to allow the entire device to better wrap around a breast. In a most preferred embodiment, the notch is configured to wrap around the nipple of the breast, and leave the nipple fully exposed. For example, the inner end of the notch can have a semi-circular shape to surround the nipple.

In a preferred embodiment, the at least one passageway is provided with an additional seam (e.g. preferably a curvilinear seam), again to increase the deformable nature of the medical device itself. The additional seam can also be utilized to restrict or limit the flow of medium contained within the passageway so that the medium remains somewhat dispersed throughout the dimensions of the medical device. For example, when the medium becomes less viscous (e.g. during heating) the additional seam(s) prevents the less viscous fluid moving downwardly during use. However, it is desirable that the additional seam does not contact the one or more seams defining the passageway so that there exists restricted flow to allow some movement of medium within the passageway.

The additional seams can be configured to restrict the flow in one direction within the passageway. For example, the medical device is provided with a plurality of semi-circular additional seams located at spaced apart positions within a single passageway, and oriented to prevent movement of the medium located in an upper position of the device to a lower position in the device. Specifically, the semi-circular shaped additional seams can be oriented to open upwardly so as to provide flow resistance in a direction opposite to that of gravity. In this manner, the medium is maintained relatively well dispersed throughout the dimensions of the medical device during use.

The medical device for treating a breast can contain a variety of mediums for heating or cooling a breast depending on the particular treatment. Preferably, a medium is selected that can provide both warm and cold therapy for treatment of tender and/or swollen breast to relieve discomfort for blocked ducts, engorgement and mastitis. A preferred medium to be used is a product known as Magic Ice.

In use, the medical device can be stored in a freezer, later removed and applied to a breast. In a different treatment, the medical device can be placed in a microwave oven, for example, at the lowest setting for thirty (30) seconds, and then the pack is removed and applied to a breast.

### BRIEF DESCRIPTION OF THE DRAWINGS

FIG. 1 shows a perspective view of a device for applying therapeutic thermal energy according to the present invention.

FIG. 2 shows a front view of the device shown in FIG. 1 being filled according to the present invention.

FIG. 3 shows a partial cross-sectional view of the device shown in FIG. 1 along the line 3-3.

FIG. 4 shows a perspective view of the device shown in FIG. 1 in use on the shoulder of a human according to the present invention.

FIG. 5 a partial perspective view of the device shown in FIG. 1.

FIG. 6 shows a front view of the device in use shown in FIG. 4.

FIG. 7 shows a perspective view of another device for applying therapeutic thermal energy according to the present invention.

FIG. 8 shows a perspective view of the device shown in FIG. 7 in use on the knee of a human according to the present invention.

FIG. 9 shows a front view of the device shown in FIG. 7 being filled according to the present invention.

FIG. 10 shows a front view of the device shown in FIG. 7.

FIG. 11 shows a side view of the device shown in FIG. 7.

FIG. 12 shows a perspective view of another device for applying therapeutic thermal energy according to the present invention.

FIG. 13 shows a perspective view of the device shown in FIG. 12 in use on the ankle of a human.

FIG. 14 shows a front view of the device shown in FIG. 12 being filled according to the present invention.

FIG. 15 shows a front view of the device shown in FIG. 12 being used at another position relative to the ankle.

FIG. 16 shows front view of the device shown in FIG. 12 being used at another position relative to the ankle.

FIG. 17 shows a front view of another device for applying therapeutic thermal energy according to the present invention.

FIG. 18 shows a perspective view of the device shown in FIG. 17 in use on the left breast.

FIG. 19 shows front view of the device shown in FIG. 17 being filled according to the present invention.

FIG. 20 shows a perspective view of the device shown in FIG. 17 in use on the right breast.

FIG. 21 is a front elevational view of a medical device for treating the breast according to the present invention.

FIG. 22 is cross-sectional view of the medical device as indicated in FIG. 21.

FIG. 23 is front elevational view of another embodiment of a medical device for treating a pair of breasts.

FIG. 24 is a further embodiment of a medical device for treating a breast.

FIG. 25 is a front elevational view of an even further embodiment of a medical device for treating the breast.

### DETAILED DESCRIPTION OF THE PREFERRED EMBODIMENTS

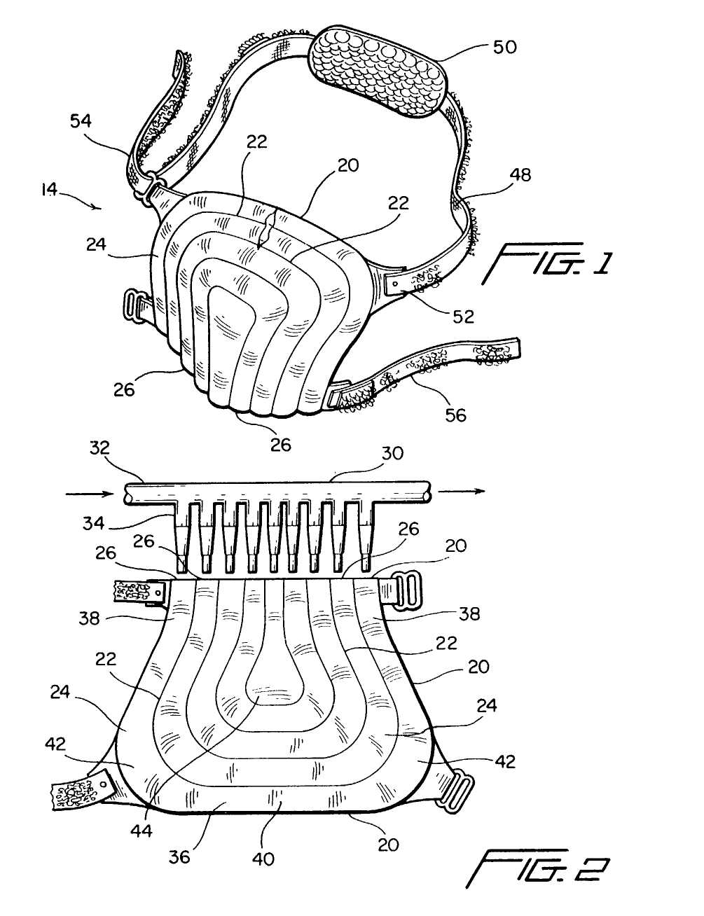

FIGS. 1-6 show a preferred embodiment of a device 14 for applying therapeutic thermal energy according to the present invention. Generally, a device 14 according to the present invention is formed from first and second layers, 16 and 18, respectively, of a deformable and resilient material, such as polyethylene sheet/strip material, which are sealingly connected along a common perimeter 20, as shown in FIGS. 1 and 3. Preferably, the sealing connection is formed from heat welding.

A device 14 according to the present invention is further characterized in that first layer 16 and second layer 18 are further sealingly connected along a plurality of, preferably congruent, curvilinear paths 22 within said perimeter 20. The sealing connection of first layer 16 to second layer 18 along paths 22 is preferably achieved by the same method as is used along perimeter 20, heat welding. Paths 22 are configured to define channels 24 having a preferably elliptical cross-section, between first layer 16 and second layer 18, as shown FIGS. 1-6. However, paths 22 may be configured to provide other configurations to each channel 24. For example, channel 24 may be configured such that each channel within layer 16 defines a radius of curvature greater than that of layer 18. This feature is particularly desirous in the event two modes of heat transfer are desired. Specifically, the layer 16 of a channel having a greater radius of curvature will tend to contact the surface of a treatment site to a greater extent, and thus apply thermal energy to the site at a greater rate, than a layer 18 of a channel having a lesser radius of curvature.

Paths 22 are further configured to provide a distance, D, between adjacent channels 24. The distance, D, between adjacent channels allows for articulation between adjacent channels, an important feature of the present invention which will be discussed further. Distance, D, also serves to ensure that adjacent channels remain preferably independent from one another such that each channel is provided with an independent channel volume. Each channel 26 is preferably at least partially filled with a medium 28 having a viscosity, preferably, greater than that of water, as shown in FIG. 3.

As shown in FIGS. 1-6, each channel 24 is provided with ends 26. At least one end 26 of each channel 24 is defined by perimeter 20 of device 14 according to the present invention. It is further preferable for the ends 26 of channels 24 defined by perimeter 20 to be adjacent one another, as shown in FIGS. 1-6. It is even further preferred for ends 26 which are defined by perimeter 20 to be even with one another, in other words, to define a plane, as shown in FIGS. 1-6.

During the manufacture of a device 14 according to the present invention, it is preferred for a pre-selected section of perimeter 20 to be maintained in an open or non-sealingly connected configuration to allow for filling of the channels 24 with a medium 28 according to the present invention. Preferably, device 14 is formed such that the section of perimeter 20 in common with the ends 26 of channels 24 is left open thereby allowing each channel to be filled individually according to the present invention.

Each channel 26 of device 14 is shown being filled with a medium 28 according to the present invention in FIG. 2. Specifically, device 14 is held such that the open preselected section 30 of perimeter 20 is maintained in a vertical orientation relative to a manifold 32. Manifold 32 is provided with nozzles 34 configured for filling each channel 24 with a medium 28 according to the present invention. Note that nozzles 34 may be configured to provide different types of medium 28. For example, it may be desirable to fill some channels with a first medium having a first specific heat and a first viscosity characteristic and some channels with a second medium having a second specific heat and a second viscosity characteristic. After filling, pre-selected section 30 is sealingly connected, preferably by heat welding, to complete perimeter 20 and the construction of device 14.

Filling of device 14 is greatly facilitated if each channel 24 is characterized by a fill constant according to the present invention. Specifically, channels 24 are preferably constructed such that the independent channel volume for each channel divided by the total number of ends 26 of that channel in common with the pre-selected perimeter section 30 is constant. A fill constant allows for nozzles 34 to have the same flow rate and thus uniform filling of each channel 24 is accomplished simply by activating manifold 32 for a duration of time. It is important to note that this feature as well as the aforementioned features of a device according to the present invention allow for high speed, low cost manufacturing.

The perimeter 20 and paths 22 of a device 14 according to the present invention are preferably configured to allow the device to adapt easily to the surface contours of the anatomy where treatment is desired. Device 14 shown in FIGS. 1-6 is configured for application of thermal energy to the shoulder. Specifically, paths 22 of device 14 are configured to provide a plurality of channels 24, each having a base portion and a pair of extending portions which extend substantially in the same direction, as shown in FIG. 2. Preferably, each base portion 36 is further characterized by a straight portion 40 and a pair of opposed acute angles 42, as is further shown in FIG. 2. Device 14 further includes a pleat 46. Preferably, pleat 46 is formed by heat welding a pair of equivalent length sections of perimeter 20, as shown in FIG. 5. Pleat 46 allows device 14 to transition from the somewhat spherical contour of the shoulder to the more cylindrical contour of the collar bone region of the body. Device 14 also includes a center channel 44 having only a single end 26 defined by preselected perimeter portion 30.

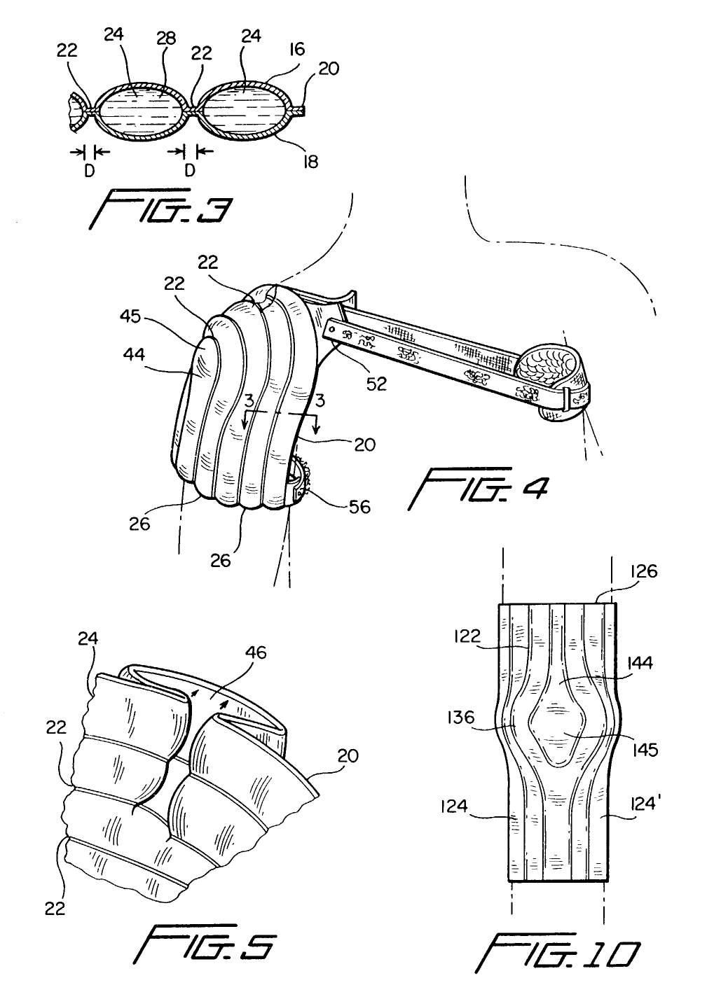

Device 14 is shown in use on the shoulder of an individual in FIGS. 4 and 6. It is important to note the manner in which paths 22 allow each channel 24 to adjust according to the topography of the shoulder. Moreover, paths 22 cause the medium within each channel 24 to be maintained uniformly throughout device 14, and thus, a uniform application of thermal energy to the treatment site. The base portion 45 of center channel 44 is somewhat enlarged to provide device 14 with the surface area and thermal capacity necessary to adequately apply thermal energy to the deltoid region of the shoulder.

Device 14 is shown secured to the body by a pair of straps. Specifically, a first strap 48 includes a first end 52 connected to an upper rear portion of device 14, a pad 50 movably secured thereto, and a second end 54 releasably connectable, preferably by hook and loop material, to an upper front portion of device 14, as shown in FIGS. 1, 4 and 6. A second strap 56 is configured similarly for securing the device to the arm of the wearer. It is important to note that the second end 54 of first strap 48 may always be maintained oriented to the front of the wearer, and thus, easily accessed, because device 14 is reversible. In other words, either layer 16 or 18 may be placed against the treatment site.

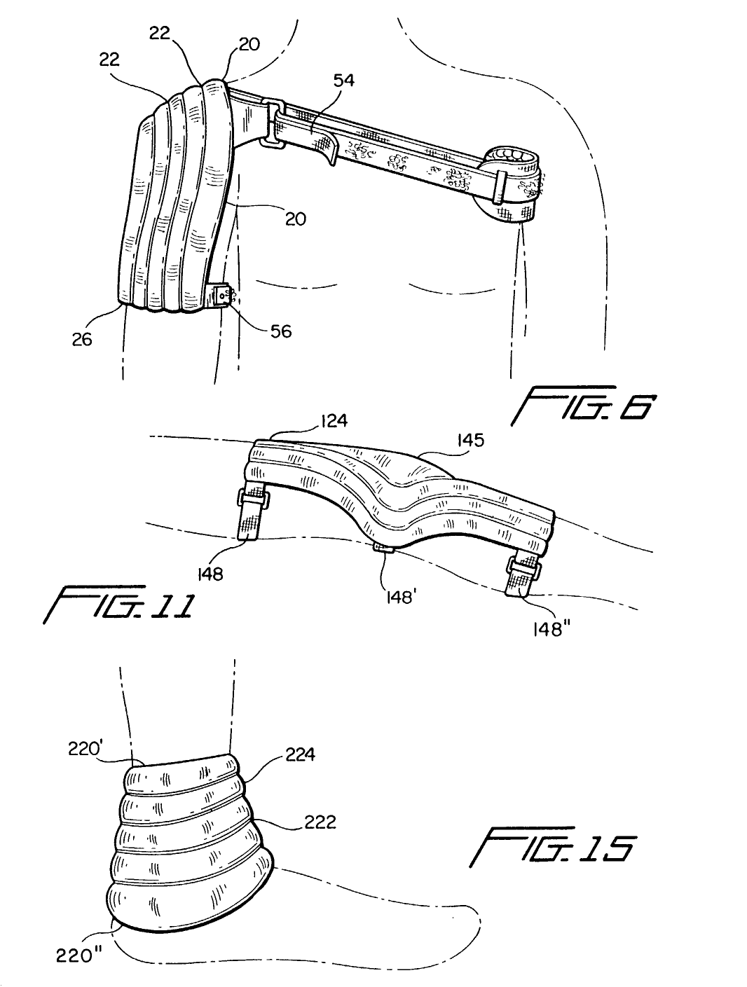

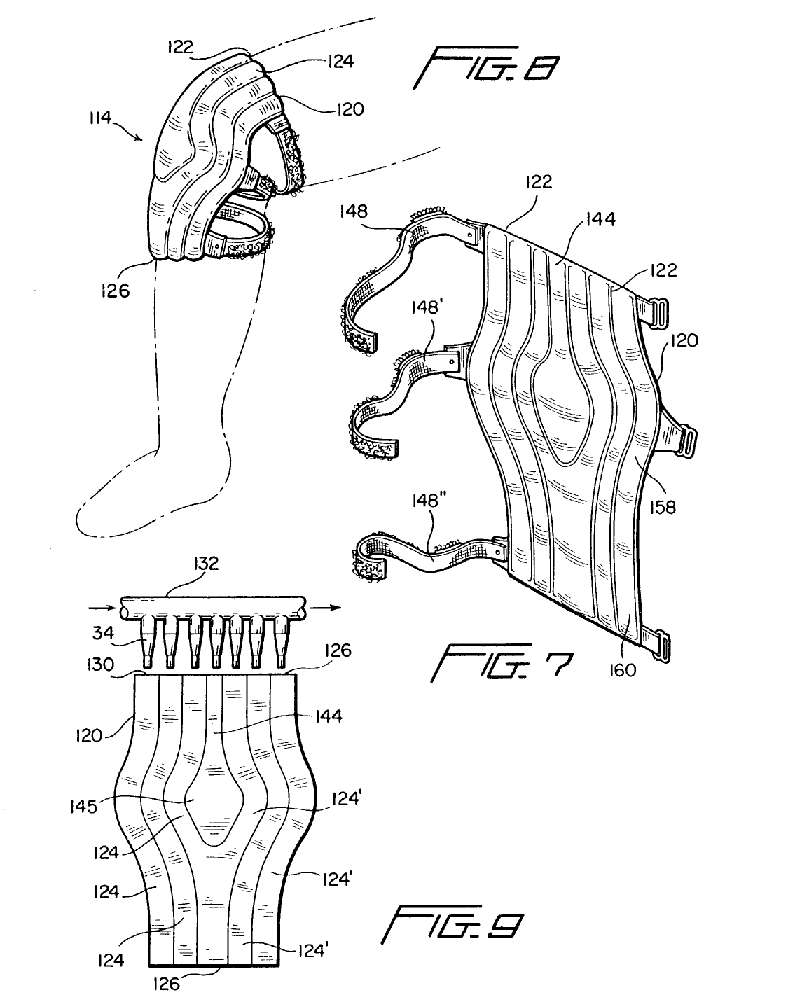

FIGS. 7-11 show a device 114 according to the present invention for applying therapeutic thermal energy to the knee. Paths 122 are configured to provide first and second sets of substantially congruent channels 124 and 124′, respectively. Each channel includes a base portion 136 having a radius of curvature 158 and a pair of extending portions 160 which extend substantially in opposite directions. First set of channels 124 and second set of channels 124′ are preferably symmetrical about a longitudinal centerline of device 114 as shown in FIG. 9. Device 114 preferably includes a center channel 144 which is provided with an enlarged base portion 145 for accommodating the patella region of the knee as shown in FIGS. 10 and 11. Center channel 144 also provides device with a larger upper portion for accommodating the typically larger circumference of the thigh, whereas the smaller lower portion of the device easily accommodates the shin or calf of the leg, as shown in FIG. 10.

FIG. 9 shows device 114 being filled according to the present invention. Specifically, the pre-selected section 130 of device 114 is shown oriented to nozzles 134 of manifold 132. Note that a single manifold and nozzle apparatus may be used to fill any number of devices according to the present invention.

Device 114 is shown being worn on the knee in FIGS. 10 and 11. Note that device 114 includes a first strap 148 for securing the device to the leg above the knee, a second strap 148′ configured for securing the device to the leg at the knee and the a third strap configured for securing the device to the leg below the knee.

FIGS. 12-16 show a device 214 according to the present invention for applying therapeutic thermal energy to the ankle. Paths 222 are configured to provide a set of substantially congruent channels 224 each having a substantially identical radius of curvature. Device 214 is provided with an upper perimeter portion 220′ and a lower perimeter portion 220″. Each channel 224 is configured to become successively longer from upper perimeter portion 220′ to lower perimeter portion 220″.

FIG. 14 shows device 214 being filled according to the present invention. Note that a pair of straps 248 and 248′ are preferably attached to the perimeter 220 of device 214 after device 214 is filled.

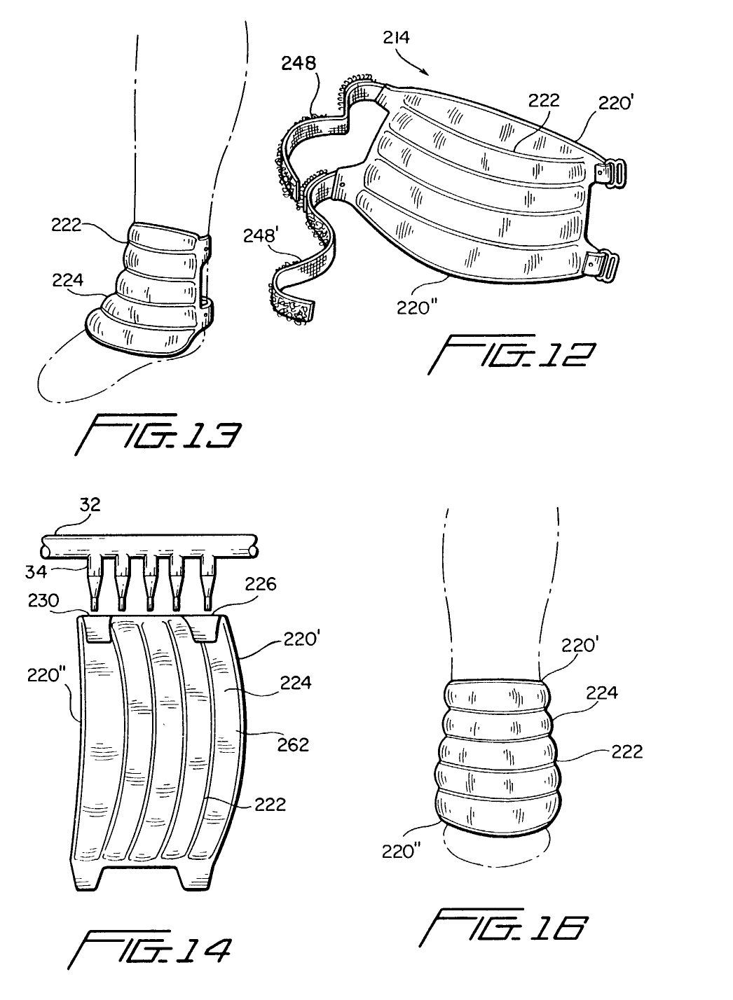

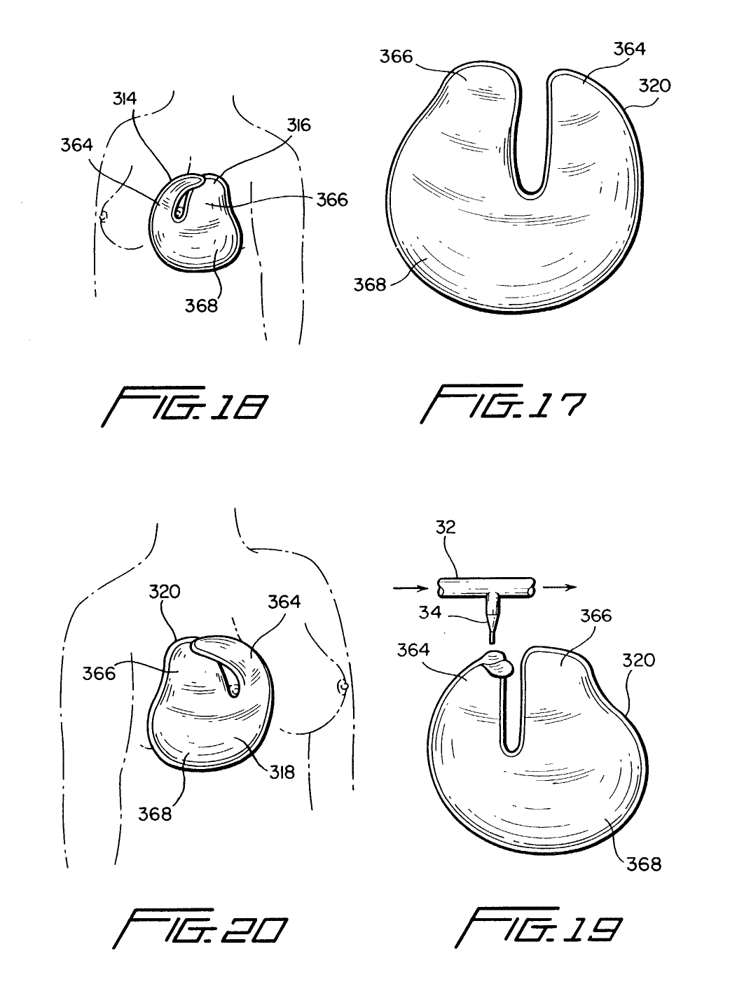

A typical use of device 214 is shown in FIG. 13. Specifically, device 214 is secured such that the surface area of the top surface of the foot and front surface of the shin are contacted by the device as shown in FIG. 13. It is important to note however that the configuration of device 214 provides versatility to the manner in which device 214 is secured to the ankle. Specifically, the configuration of device 214 allows device to be secured to the side of an ankle, as shown in FIG. 15 or to the Achilles tendon region of the ankle, as shown in FIG. 16.

FIGS. 18-20 show a device 314 for applying thermal energy to the breast according to the present invention. Device 314 provides a preferably deformable and resilient first layer 316 sealingly connected to a deformable and resilient second layer 318 along a common perimeter 320. Layers 316 and 318 define a volume which is at least partially filled with a medium having a viscosity according to the present invention as shown in FIG. 19. Layers 316 and 318 further define a first half circle shape section 364 and a second half circle shaped section 366 as shown in FIGS. 17-20.

Device 314 is shown being worn according to the present invention in FIGS. 18 and 20. Device 314 includes a bifurcation 370 which preferably originates from a center of the device and extends substantially upwardly between first section 364 and second section 366, as shown in FIG. 17. Bifurcation 370 allows a nipple of the human breast to remain substantially free of contact with device 314. Moreover, bifurcation 370 allows first section 364 to partially overlap second section 366 as shown in FIGS. 18 and 20 to produce a conical and, thus, substantially breast congruent, configuration.

It is important to note that second section 366 of device 314 is provided with an extending section 368 which extends substantially downwardly and sideways, as shown in FIGS. 18 and 20. This section is particularly useful for treating a region of the breast which is somewhat remote from the main portion of the breast and moreover, extending region 368 provides a device 314 which uniformly stands off a bra or other undergarment from the surface of the breast.

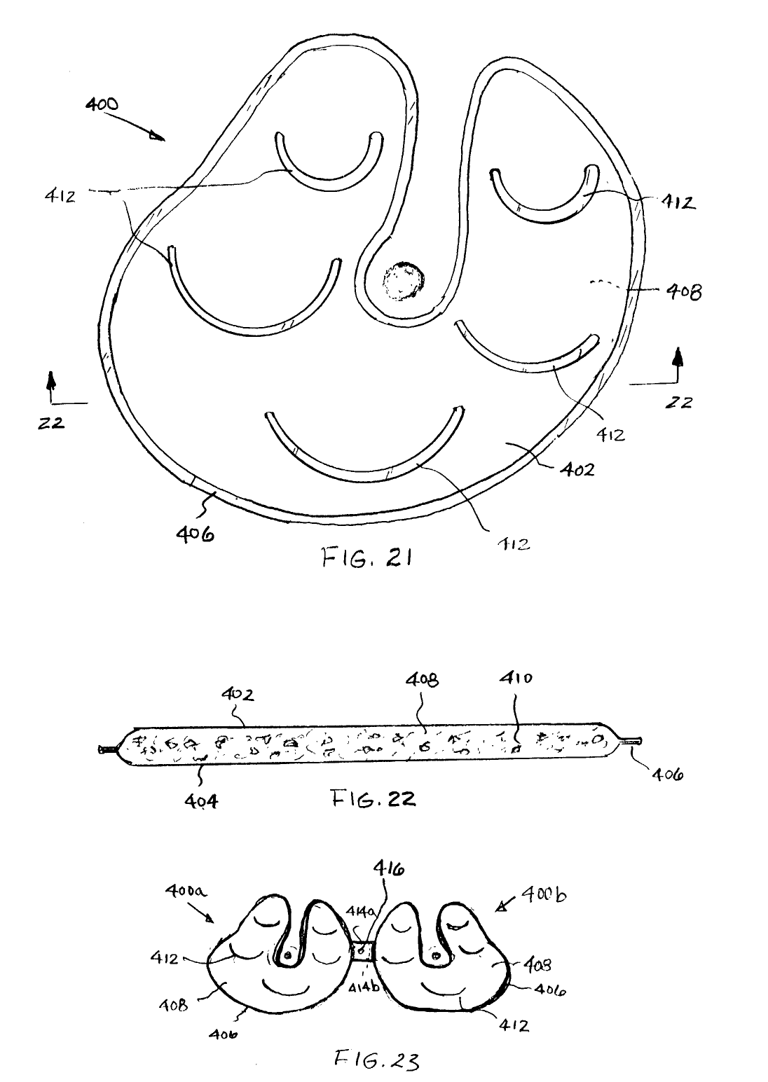

Another embodiment of a medical device 400 for treating the breast is shown in FIGS. 21 and 22.

The medical device 400 for treating a breast includes an upper layer 402 and a lower layer 402 connected together by outer perimeter seam 406. The layers 402 and 404, for example, can be made from a hypoallergenic medical-grade polyvinyl chloride (PVC), preferably that is latex-free. The seam 406 can be made by a heat sealing method or process, for example, using a heated die/cutter, which cuts and seals the outer perimeter seam 406 in a one (1) step process. It is to be noted that the outer perimeter seam 406 is curvilinear-shaped, and defines a curvilinear-shaped passageway 408 located between the upper layer 402 and lower layer 404, as shown in FIG. 22. Thus, the curvilinear-shaped nature of the outer perimeter seam 406 defines the curvilinear-shaped nature of the passageway 408, as shown in FIG. 21.

The passageway 408 is filled with a medium 410 selected to be heated or cooled prior to application or use on a breast. The medium, for example, can be a heat transfer type filler known as Magic Ice.

Preferably, semi-circular seams 412 are provided within the curvilinear-shaped passageway 408 to provide flow restrictors against the movement of the medium 410 from an upper position in the medical device 400 to a lower position therein. Specifically, the semi-circular shaped seams 412 are oriented to open in an upward position to function as one-way flow restrictors from movement of fluid from the top of the medical device 400 to the bottom of the medical device 400 (i.e. function as cupping type flow restrictors). Alternatively, other suitable shapes or configurations of the seams 412 can be made, again to function as flow restrictors and maintain the medium 410 dispensed throughout the dimensions of the medical device 400 during use.

As shown in FIG. 23, medical devices 400a and 400b can be configured to releasably connect together via tabs 414a and 414b (e.g. provided with a snap fastener 416) to provide a unit for treating a pair of breasts simultaneously. Alternatively, the medical devices 400a and 400b can be separated by undoing the snap fastener 416 and then be used to treat a single breast at a time.

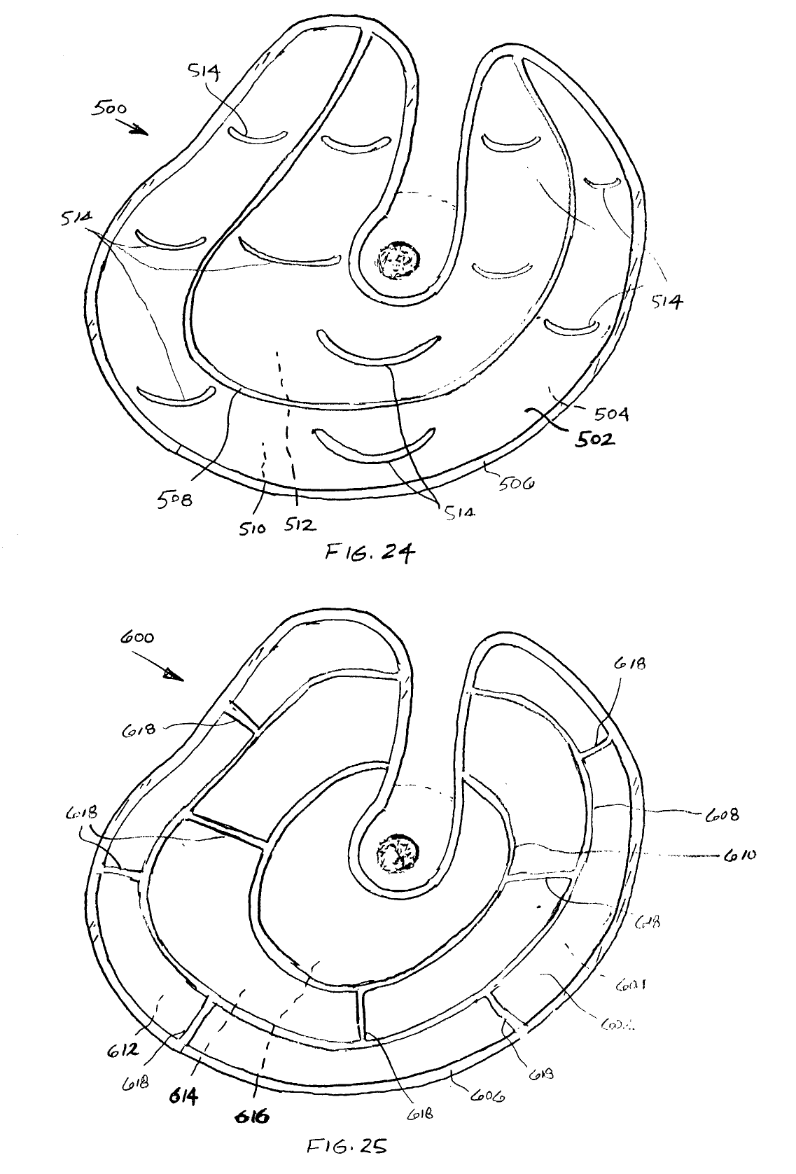

A further embodiment of a medical device 500 for treating a breast is shown in FIG. 24.

The medical device 500 is provided with an upper layer 502 and lower layer 504 connected together by an outer perimeter seam 506. In this embodiment, an inner curvilinear-shaped seam 508 is provided to define a pair of adjacent curvilinear-shaped passageways 510 and 512. The addition of the inner curvilinear-shaped seam 508 provides added deformability of the medical device 500 to further conform to the shape of a breast. The passageways 510 and 512 are provided with a plurality of semi-circular seams 514, again to act as one-way flow restrictors against the flow of medium from the upper portion of the medical device 500 to the lower portion of the medical device 500.

An even further embodiment of a medical device 600 for treating a breast is shown in FIG. 25.

The medical device 600 includes an upper layer 602 connected to a lower layer 604 by outer perimeter seam 606. The medical device 600 is provided with a pair of curvilinear-shaped inner seams 608 and 610 defining three (3) curvilinear-shaped passageways 612, 614 and 616 to further render the medical device 600 more deformable to conform to the shape of a breast. The passageway 612,614 and 616 are provided with transverse seams 618 to isolate pockets or reservoirs of the passageways 612, 614 and 615 with the heating/cooling medium to prevent migration throughout the dimensions of the medical device 600.

What is claimed is:

1. A medical device for treatment of a human or animal breast, said device comprising:
a first deformable layer;
a second deformable layer, said second layer being connected to said first layer in a manner to provide at least one curvilinear shaped seam between said first layer and said second layer to define at least curvilinear shaped one passageway between said first layer and said second layer, and configured to facilitate bending of said device to a contour of the breast;
and a medium provided within said at least one passageway for applying or removing heat energy to or from the breast.

2. A device according to claim 1, wherein said at least one seam defines an outer perimeter of the device.

3. A device according to claim 2, wherein said outer perimeter is substantially circular in shape.

4. A device according to claim 3, wherein said device is sized to cover only a portion of the breast.

5. A device according to claim 3, wherein said device is size to cover substantially the entire breast.

6. A device according to claim 1, wherein said device is configured to treat only one breast

7. A device according to claim 1, wherein said device is configured to treat a pair of breasts.

8. A device according to claim 1, wherein said device is provided with at least one notch configured to facilitate bending of the device to the contour of the breast.

9. A device according to claim 8, wherein said notch extends from an outer perimeter of the device to a center portion of the device.

10. A device according to claim 9, wherein said notch is configured to accommodate a nipple of the breast.

11. A device according to claim 10, wherein said notch is configured to allow the nipple of the breast to be left uncovered.

12. A device according to claim 3, wherein the device is provided with at least one notch configured extending from an outer perimeter of the device to a center portion of the device, said notch configured to accommodate a nipple of the breast.

13. A device according to claim 12, wherein an end of said notch is semi-circular shaped to surround the nipple of the breast.

14. A device according to claim 1, wherein said at least one seam defines an outer perimeter of the device, and including at least one additional seam located within said at least one passageway.

15. A device according to claim 14, wherein said additional seam is a curvilinear shaped seam.

16. A device according to claim 14, wherein said additional seam is located so as to not block said at least one passageway.

17. A device according to claim 15, wherein said additional seam is located so as to not block said at least one passageway.

18. A device according to claim 14, wherein said additional seam does not extend to said outer perimeter seam.

19. A device according to claim 18, wherein said additional seam is semi-circular shaped.

20. A device according to claim 19, wherein said semi-circular shaped seam is oriented to open upward.

21. A device according to claim 14, including multiple semi-circular shaped seams located within said at least one passageway, and oriented to open upward.
